(19) United States
(12) Patent Application Publication
(10) Pub. No.: US 2026/0287553 A1
(43) Pub. Date: Sep. 24, 2026

(54) MULTIMODAL PROCESSING OF PULSED EDDY CURRENT RESPONSE SIGNAL FOR WELL INTEGRITY EVALUATION

(71) Applicant: Schlumberger Technology Corporation, Sugar Land, TX (US)

(72) Inventors: Emmanuel Legendre, Clamart (FR); Isabelle Marie Dubourg, Clamart (FR); Fabrice Mege, Clamart (FR); Jean-Luc Le Calvez, Clamart (FR); Thilo Michael Brill, Clamart (FR)

(21) Appl. No.: 19/084,952

(22) Filed: Mar. 20, 2025

(51) Int. Cl. G01N 27/90; E21B 47/00; E21B 47/007; E21B 47/08; G01B 7/06; G01N 33/2045

(52) U.S. Cl. CPC G01N 27/9046; E21B 47/006; E21B 47/007; E21B 47/08; G01B 7/10; G01N 33/2045

(57) ABSTRACT

Certain aspects of the present disclosure provide a system for eddy current testing in a well. The system includes one or more transmitter coils in the well configured to generate an excitation current and one or more receiver coils in the well configured to measure an eddy current response signal from a plurality of pipes in the well. The system includes one or more processors configured to generate a time-domain waveform; determine a plurality of waveform lobes of the time-domain waveform, wherein each of the plurality of waveform lobes is associated with at least one of the plurality of pipes and with a different time interval of the time-domain waveform; and estimate one or more parameters of at least one pipe of the plurality of pipes based on one or more waveform lobes, of the plurality of waveform lobes, associated with the at least one pipe.

### BACKGROUND

### Field of the Disclosure

Aspects of the present disclosure relate to well integrity evaluation and, more particularly, to techniques for well integrity evaluation using eddy currents.

### Description of Related Art

In the field of oil and gas exploration, the efficient extraction of hydrocarbon resources is critical for maximizing production and minimizing costs. As geographic locations are increasingly developed for the extraction of hydrocarbons, the proportion of hydrocarbon production coming from mature wells also increases. Therefore, maximizing the productivity and efficiency of existing wells, in turn, becomes increasingly important.

Well intervention is one approach to increasing productivity. At some point in the life of all oil and gas wells, parts will require intervention for maintenance, repair, or replacement. During light interventions, technicians lower tools or sensors into a live well while pressure is contained at the surface. In heavy interventions, the rig crew may stop production at the formation before making major equipment changes.

Well service personnel typically perform light interventions using slickline, wireline, or coiled tubing. These systems allow operators to minimize the possibility of potential well blockages. Operators also order light interventions to change or adjust downhole equipment such as valves or pumps, or to gather downhole pressure, temperature, and flow data. Heavy interventions—also referred to as workovers—may require the rig crew to remove the wellhead and other pressure barriers from the well to allow full access to the wellbore. These operations may require a rig to remove and reinstall the wellhead and completion equipment. Heavy interventions may be used to replace parts such as tubing strings and pumps that cannot be retrieved through light interventions. Some heavy interventions are performed to plug and abandon an original producing zone to reconfigure the well to produce from a secondary zone; these operations are known as recompletions.

Issues like cracks, corrosion, fatigue, pitting, erosion, wear, and defects in components, such as pipes, casing, tubing, coatings, and welds, may arise from a combination of mechanical, chemical, and environmental factors. Repeated pressure cycles, thermal fluctuations, and vibrations can lead to fatigue and cracking, while exposure to corrosive substances (such as water, carbon dioxide, and hydrogen sulfide) and harsh environmental conditions (like saltwater or humidity) leads to corrosion and pitting. High-velocity fluid flows, especially when carrying abrasive particles, cause erosion and wear, manufacturing inconsistencies, or improper application of protective coatings, and can result in variations in material properties and defects in welds.

These issues can pose serious risks, including catastrophic equipment failure that may lead to environmental contamination and significant safety hazards for personnel. If left unchecked, cracks, corrosion, and other defects can escalate rapidly, resulting in unplanned shutdowns, costly repairs, and potentially life-threatening accidents. Regular evaluation and timely repairs are essential to maintain system integrity, ensure regulatory compliance, and protect both human lives and the environment.

Various tests may be conducted to evaluate the well, including logging tools that measure rock properties and fluid sampling. Eddy current testing is a widely used non-destructive evaluation (NDE) method that leverages electromagnetic induction to assess the integrity and material properties of metallic components, such as casing strings, pipelines, tubing, and drilling equipment. Eddy-current analysis uses electromagnetic measurements to detect, characterize, and quantify defects or thinning in metallic casing strings without requiring physical access to each casing layer.

Eddy current testing involves passing an alternating current through a transmitter coil. When an electrical current flows through a coil, a time-varying magnetic field is generated. This magnetic field induces circulating currents—known as eddy currents—in conductive materials (such as metals) near the coil. The induced eddy currents create their own secondary magnetic fields. The behavior of these eddy currents is influenced by the material's electrical conductivity, magnetic permeability, and geometric characteristics (including thickness), thereby providing a sensitive means to detect defects such as cracks, corrosion, and thinning. Changes in the secondary magnetic fields induce a voltage in a receiver coil. Analysis of the voltage response can be performed to determine characteristics of the conductive material under inspection.

One type of eddy current testing is frequency-domain eddy current testing. The system is excited with a continuous sinusoidal signal or a combination of sinusoidal signals (multi-sine or chirp signals) over a broad range of frequencies (e.g., a low frequency selected in a range 1-100 Hz). The transmitter coil generates a harmonic magnetic field that induces azimuthal eddy currents in the test material, and the resulting response is measured by one or more receiver coils. The output signal is then analyzed in the frequency domain, typically by calculating the mutual impedance or the transfer function, which relates the voltage response to the excitation current. The mutual impedance is sensitive to both the electromagnetic properties and the geometric parameters of the pipes. This approach allows for the detection of anomalies by observing changes in the amplitude and phase of the measured signal at various frequencies, with different frequencies probing different depths of the material.

Time-domain eddy current testing, by contrast, utilizes a pulsed or step excitation to induce eddy currents, capturing the transient response of the system over time. Upon the application of a step current, the induced magnetic field and subsequent eddy currents evolve dynamically, and their decay characteristics are recorded. This transient response contains rich information about the material properties and defect geometry, particularly at later times when the influence of direct coupling has diminished.

Advanced eddy-current tools use multiple frequencies of electromagnetic waves that can penetrate multiple layers of casing. By analyzing the signals, it is possible to differentiate between casings and detect defects in specific layers.

One type of time-domain eddy current testing is pulsed eddy current (PEC), enhancing depth penetration and sensitivity for multiple layers. Rather than measuring the magnetic coupling at selected discrete frequencies between a transmitter coil and several receiver coils located in the far field region, PEC logging tools use a broadband excitation and one or more sensors. Each sensor includes a transmitter coil and a receiver coil which may be mounted on the same core. The transmitter coil is excited by a short, high-energy pulsed excitation (or a step current or square pulsed current) is applied to the transmitter coil, which excites a wide spectrum of frequencies simultaneously. This broadband excitation enables rapid and sensitive probing of the test material, allowing the system to capture both surface and subsurface characteristics within a single pulse. The PEC system records a time-dependent induced voltage response (e.g., transient signal) in the receiver coil and analyzes the response signal to determine information about characteristics and geometry of the conductive material being tested.

Different sensors (i.e., a transmitter coil and receiver coil pair) of different lengths (i.e., different distances between the transmitter coil and receiver coil, e.g., in a range of 1-20 inches) have different penetration depths of investigation measurement. Thus, the use of multiple sensors simultaneously may allow for the inspection of multiple nested pipes in a well.

Both frequency-domain and time-domain eddy current testing techniques have distinct advantages, and the choice between them often depends on the specific requirements of the inspection task. Frequency-domain methods are typically favored for their simplicity and robustness in characterizing steady-state responses, while time-domain methods offer improved sensitivity to transient phenomena and subsurface defects.

One disadvantage of frequency-domain eddy current testing is that the voltage response measurements are usually displayed as wiggles, which are not easily interpretable even qualitatively.

On the other hand, one disadvantage of time-domain eddy current testing is that the voltage response waveform is typically analyzed with respect to a reference waveform, acquired at a point selected among the data and arbitrarily considered as representative of a measurement of a non-corroded portion of the material under inspection. The times at which differences appear in the voltage response waveform with respect to the reference waveform indicate variations in the thickness of the material under inspection.

In the case of a nested pipe configuration (e.g., concentric casings in the well completion), a difference appearing at an early time in the waveform is indicative of a variation of metal in the casings of small diameter (e.g., an inner casing of the nested casing configuration) and a difference appearing only at a later time in the waveform is indicative of a variation of an outer pipe of larger diameter (e.g., an outer casing of the nested casing configuration). Further, a positive variation of the voltage response waveform indicates a slower decay as compared to the reference waveform, corresponding to an increase of metal thickness. A negative variation of the voltage response waveform indicates a faster decay as compared to the reference waveform, corresponding to a decrease in the thickness (i.e., metal loss). However, when thickness variations occur simultaneously on the different nested pipes, the individual thicknesses cannot be correctly identified because the time signature on the response waveforms will overlap.

Therefore, time-domain eddy current testing relies on fragile and user-dependent relative measurements and, in some cases, cannot be used for identifying corrosion in nested pipe configurations.

In addition, the coupling between the transmitter coil and the receiver coil(s) consists of both direct and indirect components. Indirect coupling involves the interaction of the transmitter coil's magnetic field with the material under inspection. Direct coupling occurs between the transmitter coil and the receiver coil(s) without involving the test material. The direct coupling may be due to incorrect positioning of the sensor with respect to the test material. The direct coupling is primarily inductive and can become dominant when the physical spacing between the transmitter coil and the receiver coil(s) is short, potentially overwhelming the useful signal. For example, when the transmitter coil is energized with an excitation current, the transmitter coil immediately generates a strong magnetic field which can induce voltage in the receiver coil before significant eddy currents have formed in the test material. Thus, the early-time response of the system is primarily dominated by the direct coupling. The eddy currents take time to diffuse, interact with the tested material's properties, and generate the secondary magnetic fields. Thus, the late-time response of the system may be primarily dominated by the indirect coupling, and is more useful for characterization of the material and the geometry of the material.

Accordingly, only the later portions of the signal (e.g., the late times of the voltage response waveform) may be used because the earlier portions of the signal (e.g., the early times of the waveform) may be dominated by the direct sensor coupling and parasitic effects (e.g., due to sensor misalignment) which are mainly inductive and not affected by conductivity losses in the tested metal. Therefore, it is desirable to remove the direct coupling from the voltage response waveform data so that earlier portions of the signal may be useful.

Accordingly, what is needed is techniques and apparatus for improved eddy-current testing.

### SUMMARY

Certain aspects of the present disclosure provide a system for eddy current testing in a well. The system includes one or more transmitter coils in the well configured to generate an excitation current. The system includes one or more receiver coils in the well configured to measure an eddy current response signal from a plurality of pipes in the well. The eddy current response signal comprises a voltage response. The system includes one or more processors configured to determine a frequency-domain transfer function based on a ratio of the voltage response to the excitation current. The one or more processors are configured to generate a time-domain waveform based on the frequency-domain transfer function. The one or more processors are configured to determine a plurality of waveform lobes of the time-domain waveform. Each of the plurality of waveform lobes is associated with at least one of the plurality of pipes. Each of the plurality of waveform lobes is associated with a different time interval of the time-domain waveform. The one or more processors are configured to estimate one or more parameters of at least one pipe of the plurality of pipes based on one or more waveform lobes, of the plurality of waveform lobes, associated with the at least one pipe.

Certain aspects provide a method for eddy current testing in a well. The method includes generating, via one or more transmitter coils in the well, an excitation current. The method includes measuring, via one or more receiver coils in the well, an eddy current response signal from a plurality of pipes in the well. The eddy current response signal comprises a voltage response. The method includes determining, via one or more processors, a frequency-domain transfer function based on a ratio of the voltage response to the excitation current. The method includes generating, via the one or more processors, a time-domain waveform based on the frequency-domain transfer function. The method includes determining, via the one or more processors, a plurality of waveform lobes of the time-domain waveform. Each of the plurality of waveform lobes is associated with at least one of the plurality of pipes. Each of the plurality of waveform lobes is associated with a different time interval of the time-domain waveform. The method includes estimating, via the one or more processors, one or more parameters of at least one pipe of the plurality of pipes based on one or more waveform lobes, of the plurality of waveform lobes, associated with the at least one pipe.

Certain aspects provide a non-transitory computer readable medium comprising computer executable code for eddy current testing in a well. The computer executable code includes code for determining a frequency-domain transfer function based on a ratio of an eddy current voltage response signal to an excitation current. The computer executable code includes code for generating a time-domain waveform based on the frequency-domain transfer function. The computer executable code includes code for determining a plurality of waveform lobes of the time-domain waveform. Each of the plurality of waveform lobes is associated with at least one of a plurality of pipes. Each of the plurality of waveform lobes is associated with a different time interval of the time-domain waveform. The computer executable code includes code for estimating one or more parameters of at least one pipe of the plurality of pipes based on one or more waveform lobes, of the plurality of waveform lobes, associated with the at least one pipe.

Other aspects provide: an apparatus operable, configured, or otherwise adapted to perform any one or more of the aforementioned methods and/or those described elsewhere herein; a non-transitory, computer-readable media comprising instructions that, when executed (e.g., directly, indirectly, after pre-processing, without pre-processing) by one or more processors of an apparatus, cause the apparatus to perform the aforementioned methods as well as those described elsewhere herein; a computer program product embodied on a computer-readable storage medium comprising code for performing the aforementioned methods as well as those described elsewhere herein; and/or an apparatus comprising means for performing the aforementioned methods as well as those described elsewhere herein. By way of example, an apparatus may comprise a processing system, a device with a processing system, or processing systems cooperating over one or more networks.

The following description and the appended figures set forth certain features for purposes of illustration.

### BRIEF DESCRIPTION OF DRAWINGS

The appended figures depict certain features of the various aspects described herein and are not to be considered limiting the scope of this disclosure.

FIG. 1 depicts an example cased hole for wireless intervention, according to certain aspects.

FIG. 2 depicts an example magnitude Bode plot of the frequency-domain transfer functions of three ferromagnetic pipes, according to certain aspects.

FIG. 3 depicts an example phase Bode plot of the frequency-domain transfer functions of FIG. 2, according to certain aspects.

FIG. 4 depicts an example time-domain representation of the transfer functions of FIG. 2, according to certain aspects.

FIG. 5 depicts an example nested pipe configuration, according to certain aspects.

FIG. 6 illustrates decomposition of the example t-step responses illustrated for an example nested pipe configuration of three concentric pipes, according to certain aspects.

FIG. 7 illustrates an example of the first, second, and third lobes, the t-step response, and the estimated t-step response for various thicknesses of a pipe

FIG. 8 illustrates an example method for well integrity evaluation with eddy currents, according to certain aspects.

FIG. 9 illustrates an example system for well integrity evaluation with eddy currents, according to certain aspects.

### DETAILED DESCRIPTION

Aspects of the present disclosure provide apparatuses, methods, systems, and computer-readable mediums for well integrity evaluation with eddy current testing and analysis.

In some aspects, techniques are provided for acquiring eddy current response signals, determining a frequency-domain transfer function of the eddy current response, and determining a time-domain waveform from the frequency-domain transfer function. In some aspects, direct coupling is suppressed (e.g., removed) from the response signal in determining the time-domain waveform. In some aspects, the time-domain waveform is determined based on a product of time and a step response, the step response being the time-domain response of the system to a step excitation. In some aspects, the time-domain waveform shape is unchanged in response to changing electrical conductivity of the test material.

In some aspects, techniques are provided for determining, quantitatively, one or more parameters of pipes from the time-domain waveform. In some aspects, the time-domain waveform may be decomposed into waveform lobes, each waveform lobe being associated with at least one pipe. The pipes may be in a nested pipe configuration. Each waveform lobe may be a function of the thickness, electrical conductivity, and magnetic permeability of the at least one pipe. Further, the time interval of the waveform lobe may be associated with the at least one pipe. Accordingly, in some aspects, the thickness and/or the electromagnetic properties of different pipes in the nested pipe configuration may be estimated based on the associated waveform lobes.

In some aspects, the improved eddy current testing may be used to improve estimation of the parameters or characteristics of the test material and to improve well integrity evaluation. Improving the well integrity evaluation may help to more accurately or more quickly spot issues in the well such that the issue can be corrected to prevent risks, maintain system integrity, ensure regulatory compliance, and protect both human lives and the environment.

### Example Well

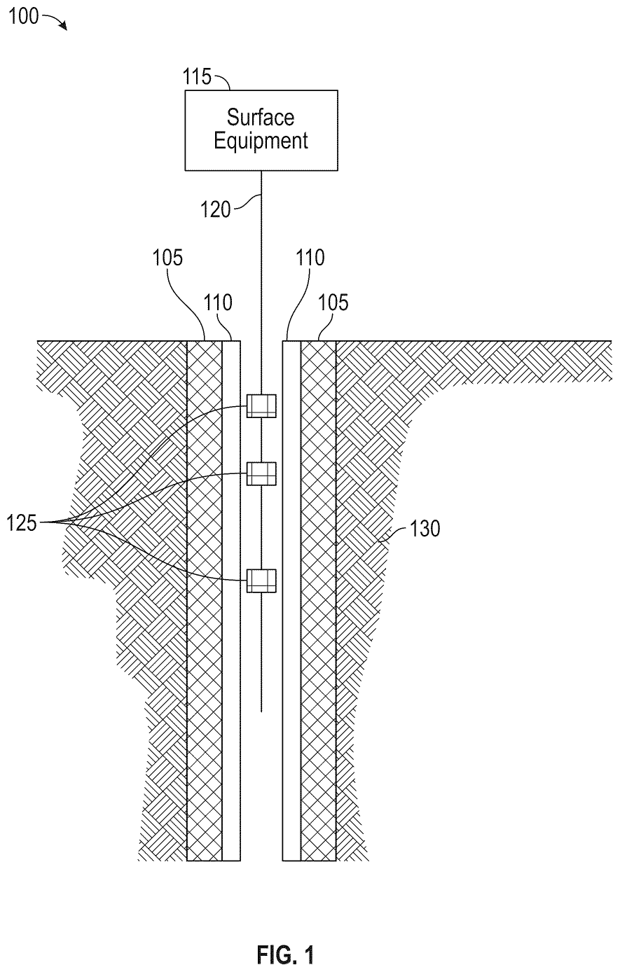

FIG. 1 depicts an example cased hole well 100 in which eddy current testing and analysis may be performed according to certain aspects. It should be understood that FIG. 1 merely depicts an example well, and that the eddy current testing and analysis techniques described herein may be performed for detection in any system.

Cased hole wells are commonly utilized in hydrocarbon extraction to enhance wellbore stability, prevent formation collapse, and improve production control. As shown, the well 100 may be drilled to a predetermined depth into a subterranean formation 130, followed by the insertion of casing 110 to isolate the formation 130 and facilitate fluid extraction. The casing string may be composed of high-strength steel or composite material. The casing 110 may be segmented into multiple sections, including surface casing, intermediate casing, and production casing, each tailored to specific depth and pressure conditions.

A cement slurry 105 may be pumped into an annulus between the formation 130 walls of the wellbore 100 and the casing 110 to secure the casing 110 in place and prevent fluid migration. The cementing process may be optimized to provide zonal isolation, enhance casing integrity, and minimize formation damage.

After cementing, a perforation system may be deployed within the casing 110 to establish communication between the wellbore 100 and the hydrocarbon-bearing formation 130. The perforation system may include shaped charges, jet perforators, or laser-based perforation tools. The perforations may be strategically placed to maximize reservoir exposure while minimizing damage to the casing and surrounding formation.

Following perforation, production tubing (not shown) may be installed to facilitate fluid extraction. The production tubing may be equipped with flow control devices, sand screens, or intelligent monitoring systems to regulate production and optimize recovery. Well stimulation techniques such as acidizing or hydraulic fracturing may be employed to enhance permeability and hydrocarbon flow.

Downhole sensors 125 and real-time monitoring equipment are integrated into the well system to assess pressure, temperature, and flow dynamics. Data analytics and machine learning algorithms can be applied to optimize production rates and predict maintenance requirements.

Wireline intervention involves deployment of tools and instruments, such as the downhole sensors 125, into the cased hole well 100 using a cable or slickline 120 to perform maintenance, logging, or remedial operations. Wireline equipment may include a cable drum, a surface control unit (e.g., surface equipment 115), and specialized downhole tools designed for various applications. Logging tools may be used to assess well integrity, measure pressure and temperature, and evaluate fluid composition.

According to certain aspects, the wireline intervention may include equipment for eddy current testing and analysis as described herein.

### Example Well Integrity Evaluation with Eddy Current Testing

Eddy current testing and analysis can be used to estimate characteristics of a material under inspection, which may be useful for evaluating well integrity.

An eddy current system or tool excites a transmitter coil with an electromagnetic signal, referred to herein as the excitation signal or excitation current. The excitation signal may excite a range of frequencies. A frequency-domain based eddy current tool may use a sinusoidal (e.g., multi-sine or chirp) excitation signal. A time-domain based eddy current tool may use a pulsed current or step current excitation signal that excites a broadband spectrum of frequencies simultaneously.

The excitation signal induces a time-varying magnetic field. This magnetic field penetrates through conductive materials and induces eddy currents in the material under inspection. The eddy currents decay over time, with the decay rate depending on material properties such as the thickness, conductivity, and magnetic permeability of the material.

The eddy currents generate secondary magnetic fields. Changes in the secondary magnetic fields induce a voltage response that is measured by the receiver coils. The voltage response signal measured by the receiver coils is referred to herein as the eddy current response signal. In frequency-domain based eddy current approaches, the eddy current response signal may be analyzed in the frequency domain. In the time-domain based eddy current approaches, the time-dependent transient voltage response is analyzed. From Faraday's Law of Induction, the magnetic vector potential will drive a voltage on the receiving coil ends through a magnetic flux variation given by the quasi-stationary eddy current equation:

$${v}_{{Γ}_{RX}} ( t ) = {∮}_{{Γ}_{RX}} E \cdot dl = - \frac{\partial }{\partial {t}_{t}} {∮}_{{Γ}_{RX}} A \cdot dl \qquad ( Eq . 1 )$$

where, E is the electric field around the receiver, ν_{Γ}_{_{Tx}}(t) is the voltage induced across the terminals of the receiver coil Γ_{RX}, [symbol] _{RX} E·dl is the line integral of the electric field around the receiver coil, and

$$- \frac{\partial }{\partial {t}_{t}} {∮}_{{Γ}_{RX}} A \cdot dl$$

is the time derivative of the loop integral of the magnetic vector potential reflecting that the voltage is induced by the changes in the magnetic field A.

In some aspects, a double-function coil is used, acting as both the transmitter coil (e.g., driver) and the receiver coil (e.g., pick-up). In some aspects, separate transmitter coil and receiver coils are used.

The induced voltage in the receiver coil depends on the strength of the eddy currents, which is influenced by material properties like thickness, electrical conductivity, and defects. For example, ferromagnetic material introduces magnetic reluctance, which impacts the inductive coupling and eddy currents. Changes in thickness or permeability of the test material will influence the amount of energy coupled into the material, as well as how much energy is reflected back to the receiver coil.

Thus, the eddy current response signal may be analyzed (e.g., using digital signal processing) to determine characteristics of the test material. In some aspects, the analysis of the eddy current response involves calculating the transfer function, which relates the voltage response to the excitation current. The transfer function represents the relationship of the input, i.e., the signal generated by the transmitter coil, and the output, i.e., the eddy current response signal detected by the receiver coil.

The transfer function is the response of the system in the frequency domain and may be converted to the time-domain using a Laplace transform or a Fourier transform. While aspects of the present disclosure are described in the Laplace domain, it should be understood that the aspects equally apply in the Fourier domain.

The characteristics of the test material are not expected change over short periods of time and, therefore, the system may be considered linear-time invariant. In a linear-time invariant system, the transfer function is characterized by its impulse response or the trans-impedance given by the Laplace transform of the continuous-time input excitation signal current i_{Γ}_{_{TX}}(t) to the transmitter coil Γ_{TX} and the Laplace transform of the output eddy current response signal voltage ν_{Γ}_{_{RX}}(t) on the receiver coil Γ_{RX}:

$$ℒ \{ {i}_{{Γ}_{TX}} ( t ) \} = {I}_{{Γ}_{TX}} ( s ) ; \qquad ( Eq . 2 )$$

$$ℒ \{ {v}_{{Γ}_{RX}} ( t ) \} = {V}_{{Γ}_{RX}} ( s ) ;$$

$$s = j \cdot ω ;$$

$$Z ( s ) = \frac{{v}_{{Γ}_{RX}} ( s )}{{I}_{{Γ}_{TX}} ( s )}$$

where, Z(s) is the trans-impedance function, s is a complex variable with an imaginary part j·ω representing an oscillatory component.

The mutual impedance is sensitive to both the electromagnetic properties and the geometric parameters of the material under inspection. The mutual impedance is complex valued with both amplitude and phase.

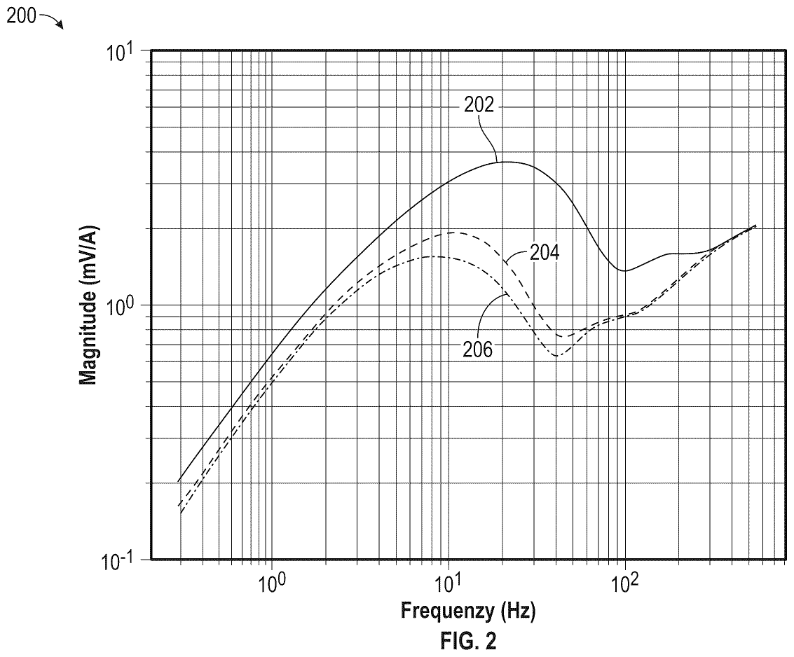

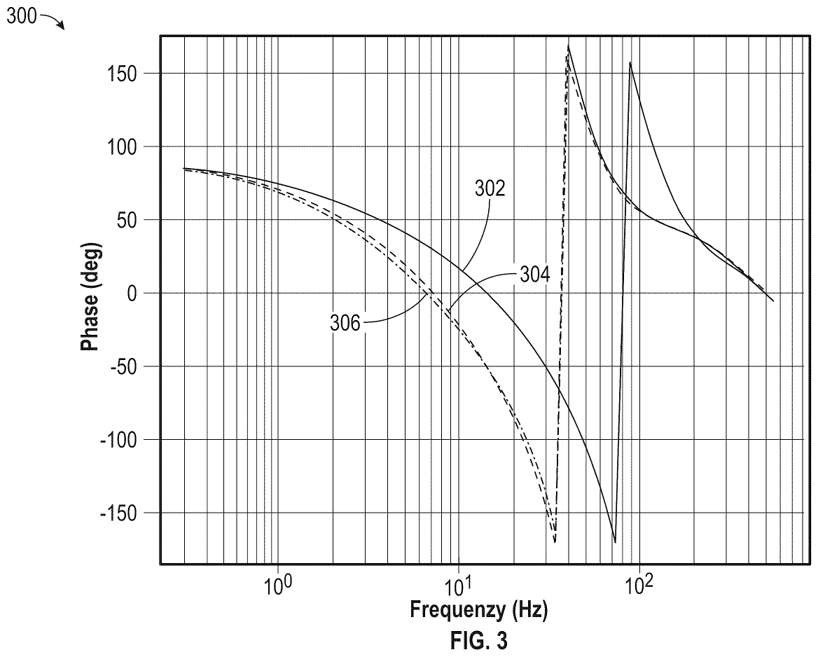

The transfer function can be depicted as a Bode plot. FIG. 2 depicts an example magnitude Bode plot 200 of the frequency-domain transfer functions 202, 204, 206 of three ferromagnetic pipes. The magnitude Bode plot 200 illustrates how the magnitude of the induced voltage changes with the frequency of the excitation signal. The magnitude Bode plot 200 may reflect the influence of material properties like thickness and magnetic permeability on inductive coupling and eddy currents. FIG. 3 depicts an example frequency-domain phase Bode plot 300 of the transfer functions 302, 304, 306 of the three ferromagnetic pipes. The phase Bode plot 300 shows the phase shift between the input excitation signal and the output eddy current response signal. The phase shift may change from inductive behavior (positive phase shift) to resistive behavior (negative phase shift) as the excitation frequency increases.

As shown in the Bode plots 200, 300, the system behaves inductively at low frequencies, with the transfer function exhibiting high gain (amplitude) and a phase shift toward +90°. The low frequency part of the transfer function is approximately proportional to the excitation frequency, for example capturing a magneto-static coupling sensitive to magnetic permeabilities and geometries of the casings. As the excitation frequency increases, the electrical conductivities of the casings start to play a role. The magnitude decreases due to ohmic losses of the eddy current, with the rate of attenuation varying based on the material's permeability and thickness. The phase would shift towards −90° as the system behaves more resistively due to eddy current formation. At high frequencies, direct couplings and simple reflection on inner-most surface dominate the measurement.

### Example Well Integrity Evaluation with Eddy Current Diffusion Time Response

As discussed herein, the transfer function incorporates the effects of inductive coupling, eddy current formation, and how the material's properties (thickness, permeability) affect the interaction. Thus, the eddy current response signal may be analyzed at different time intervals to differentiate between material layers and to detect defects or corrosion in underlying layers. Changes in amplitude of the voltage of the eddy current response signal can indicate material loss (corrosion, thinning) or variations in conductivity (due to alloy composition or temperature effects). A faster rate of decay indicates higher conductivity (since eddy currents dissipate quickly), while a slower rate of decay suggests lower conductivity or thicker material. Changes in mutual impedance may be used to assess properties of the material under inspection

However, as further discussed herein, the early-time response of the system is primarily dominated by the direct coupling, whereas the late-time response of the system may be primarily dominated by the indirect coupling, and is more useful for characterization of the test material. Aspects of the present disclosure provide techniques for removing the direct coupling from the response signal, providing an output waveform that is free of the disturbance of inductive direct coupling and, in addition, having a shape that is independent of the electrical conductivity of the material under inspection.

For the quasi-stationary eddy current approximation, the solution of the eddy current problem satisfies the diffusion equation which models how the eddy current diffuse over time due to the electrical conductivity of the test material:

$$( curl \frac{1}{μ} curl A ( x , t ) ) + σ {\partial }_{t} A ( x , t ) = J ( x , t ) in Ω \times ( 0 , T ) , \\ Ω ⊂ {R}^{3} , and T > 0 \qquad ( Eq . 3 )$$

where, A(x,t) is the magnetic vector potential as a function of space and time, μ is the magnetic permeability of the test material, σ is the electrical conductivity of the test material, J(x,t) is the excitation signal divergence-free current density as a function of space and time, Q⊂R³ represents the spatial domain, and T>0 defined the time domain of interest. The magnetic vector potential A may be known via a Coulomb gauge.

The current density of the excitation signal is given by:

$$J ( x , t ) = {i}_{TX} ( t ) \times {β}_{TX} ( t ) \times {τ}_{{Γ}_{TX}} ( t ) \qquad ( Eq . 4 )$$

where, J(x,t) is the current density, i_{TX}(t) is the excitation current via a multi-turn coil carrying the current, along a path of the transmitter coil Γ_{TX}, τ_{Γ}_{_{TX}} is the normalized tangential vector along Γ_{TX}, and β_{TX} is a coordinate dependent scalar (e.g., a spatial distribution function) of dimension 1/m² that characterizes the winding density per unit area of the coil.

The transfer function can be decomposed into two contributions:

$$Z ( s ) = M \cdot s + F ( s ) \qquad ( Eq . 5 )$$

where, M is the mutual inductance between transmitter and receiver coils, M·s represents the direct coupling component, and F(s) is the through-sample transfer function change representing the indirect coupling component.

According to certain aspects, the transfer function can be divided by s, followed by a derivation with respect to s, to suppress the mutual impedance (i.e., the direct coupling) component, resulting in:

$$H ( s ) = - \frac{\partial }{\partial s} \frac{Z ( s )}{s} = - \frac{\partial }{\partial s} \frac{F ( s )}{s} \qquad ( Eq . 6 )$$

H(s) is the Laplace transform of a time-domain function waveform h(t). For example, the Laplace transform of the Heavyside step function H(t) is:

$$ℒ \{ H ( t ) \} = \frac{1}{s} \qquad ( Eq . 7 )$$

where, H(t)=0 for t<0 and H(t)=1 for t≥0.

Multiplying a function ƒ(t) in the time domain by t corresponds to the negative derivative of its Laplace transform with respect to s:

$$ℒ \{ tf ( t ) \} = - \frac{\partial }{\partial s} ℒ \{ f ( t ) \} \qquad ( Eq . 8 )$$

Thus, mapping H(s) in the time-domain via an inverse Laplace transform provides the time-domain waveform h(t), and corresponding to the step response multiplied by time t:

$$h ( t ) = t \cdot {ℒ}^{- 1} ( \frac{Z ( s )}{s} ) \qquad ( Eq . 9 )$$

The generated time-domain waveform h(t) is immune to the inductive contribution (M·s) contained in the decomposition of Z(s). As shown in Eq. 6, by suppressing the mutual inductance, H(s) depends only on the through-sample transfer function change F and not on the mutual inductance M.

Also, using this approach, the generated time-domain response waveform h(t) has a shape that is unchanging in response to a change in the electrical conductivity σ of the material. For example, for \tilde{σ}=ασ where α>0, the corresponding magnetic vector potential Ã(x; t) solution of the quasi-stationary eddy current Eq. 3 becomes:

$$( curl \frac{1}{μ} curl \tilde{A} ( x , t ) ) + ασ {\partial }_{t} \tilde{A} ( x , t ) = \tilde{J} ( x , t ) in Ω \times ( 0 , T ) , \\ Ω ⊆ {R}^{3} , T > 0 \qquad ( Eq . 10 )$$

The solution of the quasi-stationary eddy current Eq. 10 is valid for all t∈(0,T) and, therefore, is also valid for all τ=t/α in (0,T/α):

$$( curl \frac{1}{μ} curl \tilde{A} ( x , τ ) ) + ασ {\partial }_{t} \tilde{A} ( x , τ ) = \tilde{J} ( x , τ ) in Ω \times ( 0 , \frac{T}{α} ) , \\ Ω ⊆ {R}^{3} , T > 0 , α > 0 \qquad ( Eq . 11 )$$

Given ∂ₜτ=1/α, the quasi-stationary eddy current solution becomes:

$$( curl \frac{1}{μ} curl \tilde{A} ( x , τ ) ) + σ {\partial }_{τ} \tilde{A} ( x , τ ) = \tilde{J} ( x , τ ) in Ω \times ( 0 , \frac{T}{α} ) , \\ Ω ⊆ {R}^{3} , T > 0 , α > 0 \qquad ( Eq . 12 )$$

The Eq. 12 is a time-scaled version of the quasi-stationary eddy current solution for the time interval

$$( 0 , \frac{T}{α} ) ,$$

hence the magnetic vector potential is:

$$\tilde{A} ( x , t ) = A ( x , \frac{t}{α} ) in Ω \times ( 0 , \frac{T}{α} ) , Ω ⊆ {R}^{3} , T > 0 , α > 0 \qquad ( Eq . 13 )$$

For the changed electrical conductivity σ of the material, \tilde{σ}=ασ, the induced voltage in the receiver, following the quasi-stationary eddy current equation, the induced voltage is:

$$\begin{matrix}{\tilde{v}}_{RX} ( t ) = - {\partial }_{t} {∮}_{{Γ}_{RX}} \tilde{A} ( x , t ) \cdot dl \\ = - {\partial }_{t} {∮}_{{Γ}_{RX}} A ( x , \frac{t}{α} ) \cdot dl \\ = \frac{1}{α} {v}_{RX} ( \frac{t}{α} )\end{matrix} \qquad ( Eq . 14 )$$

The excitation current corresponding to the step response is:

$${\tilde{ι}}_{TX} ( t ) = {i}_{TX} ( \frac{t}{α} ) \qquad ( Eq . 15 )$$

The generated time-domain waveform in this case is:

$$\begin{matrix}\tilde{h} ( t ) = t \cdot {\tilde{v}}_{RX} ( t ) for {\tilde{ι}}_{TX} ( t ) \\ = \frac{t}{α} \cdot {v}_{RX} ( \frac{t}{α} ) for {i}_{TX} ( \frac{t}{α} ) \\ = h ( \frac{t}{α} )\end{matrix} \qquad ( Eq . 16 )$$

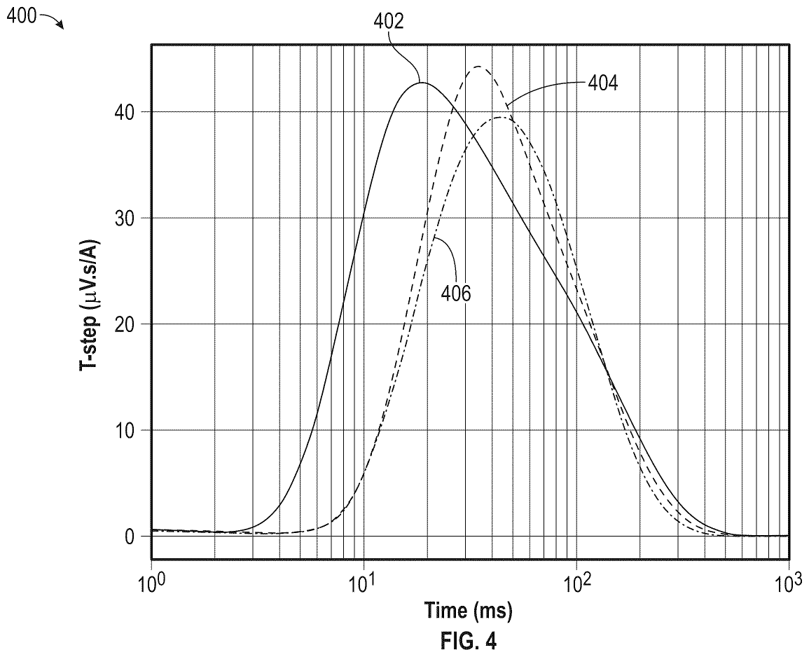

As shown in the above Eqs. 9-16, the generated time-domain waveforms corresponding to different electrical conductivities of the material are translated versions of each other on the logarithmic time axis and the shape of h(t) is unchanged in response to a change in electrical conductivity. FIG. 4 depicts the step response (t-step) waveforms 402, 404, 406 depicted versus a logarithmic timescale for the three pipes.

Accordingly, the eddy current system excites a transmitter (TX) with multi-sine, chirp, or pulse-like signals and receives a response at one or more receivers (RX). The eddy current system estimates a frequency domain transfer function Z(s) (e.g., Eq. 5) of the coupling between the transmitter TX and receiver RX. The eddy current system may compute a time-domain waveform w(t) response signal by applying an inverse Laplace transform to

$$( - \frac{d}{ds} \frac{Z ( s )}{s} )$$

or equivalently by multiplying time by the step response of the transfer function (e.g., Eqs. 6-9). The eddy current system may perform digital signal processing (e.g., signal decomposition, inversion, machine learning, etc.) using as input the generated waveform w(t) to estimate parameters of interest (e.g., electrical conductivity, magnetic permeability and geometry) of a tested material, the parameters influencing the coupling between the TX and RX.

### Example Multimodal Processing of Eddy Current Response Signal for Well Integrity Evaluation with Nested Pipes

In some systems for pulsed eddy current (PEC) non-destructive testing (NDT), the impedance is analyzed by comparison with a reference measurement. For example, the reference measurement may be a measurement performed in air (i.e., with no metal sample) and/or with a reference to a sample (e.g., a sample metal with known properties).

In the context of underground pipe evaluation, reference sample measurement has limited usefulness due to the high variability of the properties of the pipe. In such cases, reference measurement in air has more usefulness as it reveals the impedance change due to the conductive sample under test. For example, the impedance Z can be expressed as the sum of impedance in air Zair and the impedance change due to the sample under test as:

$$Z ( s ) = {Z}_{air} ( s ) + Δ Z ( s ) \qquad ( Eq . 17 )$$

Various useful features can be derived from pulsed eddy current trans-impedance of a pipe under testing. For example, the slope of the time-domain response can be derived and evaluated over a logarithmic timescale over a sufficiently large time. However, one difficulty with evaluation of multiple pipe configurations is that the unknown parameters space grows large with a greater number of pipes in the configuration. For example, the relative centering of the pipes may not be known. Therefore, techniques for evaluating measured pulsed eddy current trans-impedance of multiple (e.g., nested) pipes is needed.

Accordingly, the present disclosure provides techniques for multimodal processing of eddy current response data that can be used to evaluate nested pipes.

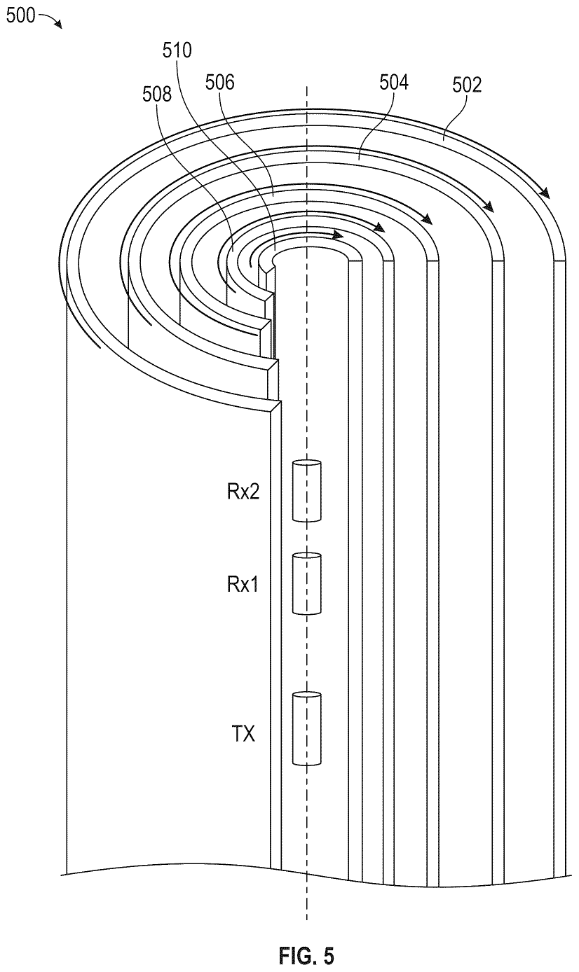

FIG. 5 depicts an example nested pipe configuration 500. As shown, the nested pipe configuration 500 includes an outer pipe 502, an inner pipe 510, and three concentric pipes 504, 506, and 508 between the outer pipe 502 and the inner pipe 510. In the example shown in FIG. 5, the system includes a transmitter coil (TX 512) and two receiver coils (RX 514 and RX 516) within the nested pipe configuration. The two receiver coils are spaced at different distances from the transmitter coil, providing for different penetration depths of the investigation.

The transmitter coil may excite multiple frequencies and covers a large bandwidth (e.g., from a fraction of Hz and up to almost a kHz). From the voltages measured at each receiver coil, the frequency-domain transfer function of the transmitter-receiver coupling is estimated. A time-domain representation can be computed from the transfer function. For example, the time-domain representation may be computed in the Laplace domain or in the Fourier domain. In some aspects, the time-domain representation, or t-step response (e.g., as illustrated in FIG. 4), can be computed as discussed above with respect to the Eqs. 6-9.

It should be understood that while aspects of the present disclosure are discussed with respect to a nested pipe configuration having three concentric pipes, the aspects discussed herein can be equally applied to any pipe configuration having any number of pipes which may be concentric or not concentric.

In some aspects, early times of the t-step response waveform may be associated with the innermost pipe while later times of the t-step response waveform may be associated with the outermost pipe. According to certain aspects, after measuring pulsed eddy current responses and determining the t-step response waveforms, the t-step response waveform may be decomposed into individual waveform “lobes”, where the mixture of the individual lobes approximates the t-step response waveform. Each individual lobe may be associated with at least one of the pipes. For example, each individual lobe is a function of the thickness of a pipe (and the thicknesses of one or more smaller pipes, such as nested pipes within) and the electromagnetic properties (e.g., electrical conductivity and magnetic permeability) of all of the pipes. Accordingly, by monitoring changes to the eddy current response over time (and the respective t-step responses), the individual lobes can also be determined, and the changes to the thickness and/or to the electromagnetic properties of each associated pipe may be estimated based on the changes to the respective lobes.

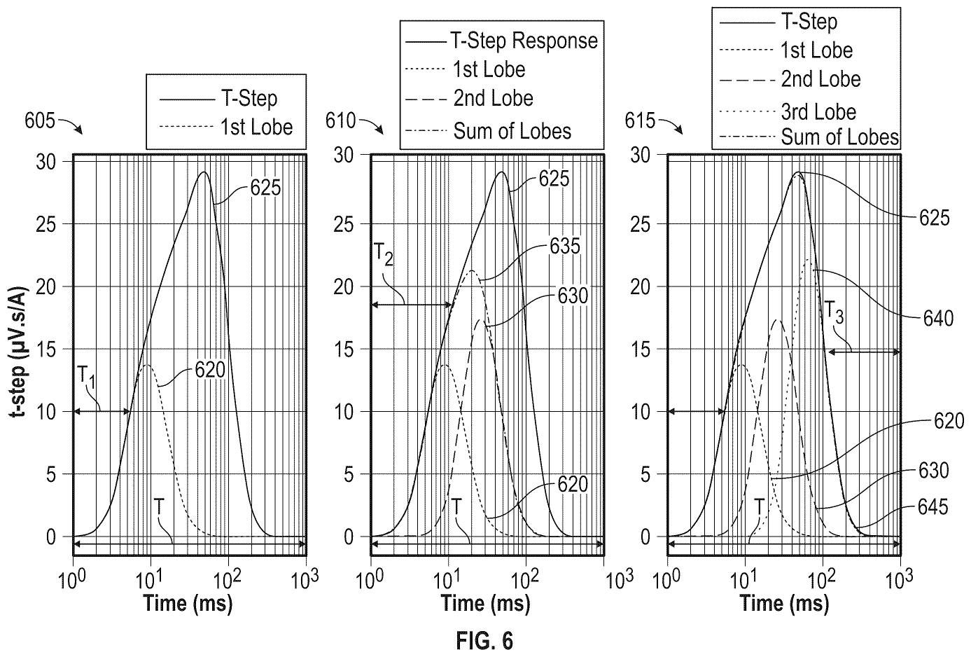

According to certain aspects, the t-step response may be decomposed into a mixture of individual lobes, as shown in the example in FIG. 6. In some aspects, the t-step response may be decomposed as a sum, a weighted sum, or a convolution of the individual lobes. In some aspects, the t-step response may be decomposed into a mixture of individual lobes corresponding to the number of pipes (e.g., the number of concentric pipes in a nested pipe configuration). In some aspects, the t-step response is fitted to the mixture of the individual lobes.

FIG. 6 illustrates decomposition of the example t-step responses illustrated for an example nested pipe configuration of three concentric pipes. The graph 605 in FIG. 6 depicts a first lobe 620 of the t-step response 625. In some aspects, the first lobe 620 may be determined as a function of the thickness, d₁, of the innermost pipe (first pipe) and the electromagnetic properties of the nested pipes. For example, the electromagnetic properties include the electrical conductivity (σ) and magnetic permeability (μ) of the innermost pipe (σ₁, μ₁) and the electrical conductivity and magnetic permeability of the second pipe (σ₂,μ₂) and third pipe (σ₃,μ₃). The t-step response 625 has a total time interval T. In some aspects, the first lobe 620 approximates an early time interval T₁ of the t-step response 625.

The graph 610 in FIG. 6 depicts the first lobe 620 and a second lobe 630 of the t-step response 625, and the sum of lobes 630 of the first lobe 620 and the second lobe 630. In some aspects, the second lobe 630 may be determined as a function of d₁, the thickness, d₂, of the next innermost pipe (second pipe) and the electromagnetic properties of the nested pipes, (σ₁,μ₁), (σ₂,μ₂), and (σ₃,μ₃). In some aspects, the sum of lobes 630, of the first lobe 620 and the second lobe 630, approximates an intermediate time interval T₂ of the t-step response 625.

The graph 615 in FIG. 6 depicts the first lobe 620, the second lobe 630, and a third lobe 640 of the t-step response 625, and the sum of lobes 645 of the first lobe 620, the second lobe 630, and the third lobe 640. In some aspects, the third lobe 640 may be determined as a function of d₁, d₂, the thickness, d₃, of the last innermost pipe (third pipe) and the electromagnetic properties of the nested pipes, (σ₁,μ₁), (σ₂,μ₂), and (σ₃,μ₃). In some aspects, the third lobe 640 approximates a late time interval T₃ of the t-step response 625. In some aspects, the sum of lobes 645 of the first lobe 620, the second lobe 630, and the third lobe 640, approximates the t-step response 625 over the full time interval T.

In a cased well (e.g., such as the example well 100), the geometrical parameters (e.g., thickness) and electromagnetic properties (e.g., conductivity and permeability) of the pipes may vary. Thus, the actual values of the parameters and properties may be unknown quantities.

According to certain aspects, the response waveform may be fitted by a sum of lobes in a coherent manner according to dependencies of the physical parameters of the pipes. According to certain aspects, as the lobes are parametrized by those unknown quantities, and the lobes can be used to reconstruct the measured t-step by minimizing a residue from the reconstructed t-step response (as a mixture of the individual lobes lᵢ(t) with [symbol] the mixing operator) to estimate the physical parameters of the pipes:

$$‖ w ( t ) - ℳ ( {l}_{i} ( t ) ) ‖ \qquad ( Eq . 18 )$$

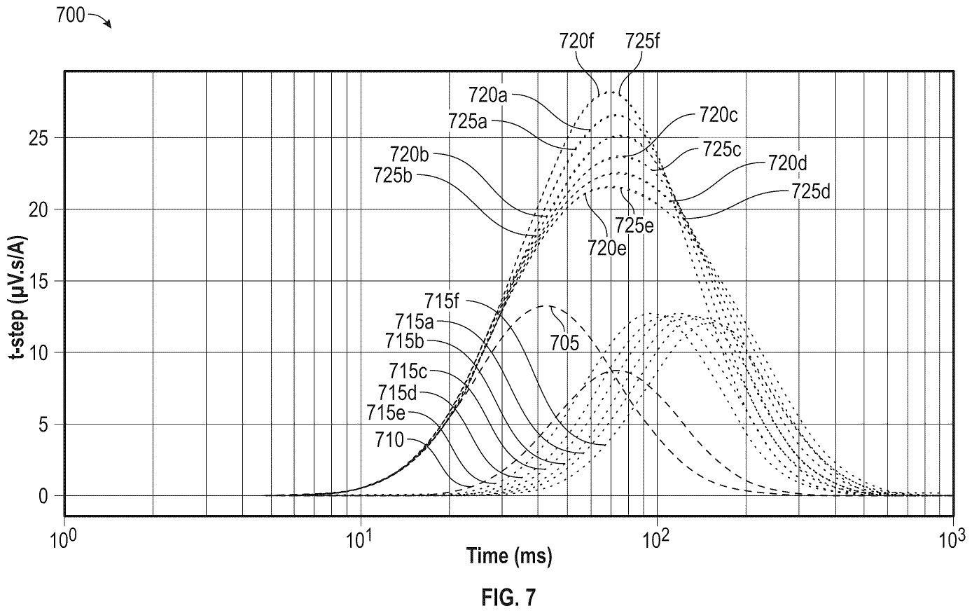

FIG. 7 illustrates an example of the first, second, and third lobes, the t-step response, and the estimated t-step response for various thicknesses of a pipe (e.g., various third outermost pipe thicknesses). The graph 700 depicts examples of the lobes and t-step responses for a third pipe having a thickness (e.g., d₃) varied to 40% (e.g., 0.4 d₃), 55% (e.g., 0.55 d₃), 70% (e.g., 0.7 d₃), 85% (e.g., 0.85 d₃), 100% (e.g., d₃), and 115% (e.g., 1.15 d₃). As shown in FIG. 7, the first lobe 705 and the second lobe 710 may remain unchanged as the thickness of the third pipe varies. As shown, the third lobe 715a (corresponding to d₃) shifts as the thickness of the third pipe decreases as seen by the 85% third lobe 715b, the 70% third lobe 715c, the 55% third lobe 715d, and the 40% third lobe 715e, and the third lobe 715a shifts as the thickness of the third pipe increases as seen by the 115% third lobe 715f. As shown, the t-step response 720 and the approximated t-step response 725 shifts as the thickness of the third pipe decreases as seen by the 85% t-step response 720b and the 85% approximated t-step response 725b, the 70% t-step response 720c and the 70% approximated t-step response 725c, the 55% t-step response 720d and the 55% approximated t-step response 725d, and the 40% t-step response 720e and the 40% approximated t-step response 725e. As shown, the t-step response 720a shifts as the thickness of the third pipe increases as seen by the 115% t-step response 720f and the 115% approximated t-step response 725f.

### Example Method for Well Integrity Evaluation with Eddy Currents

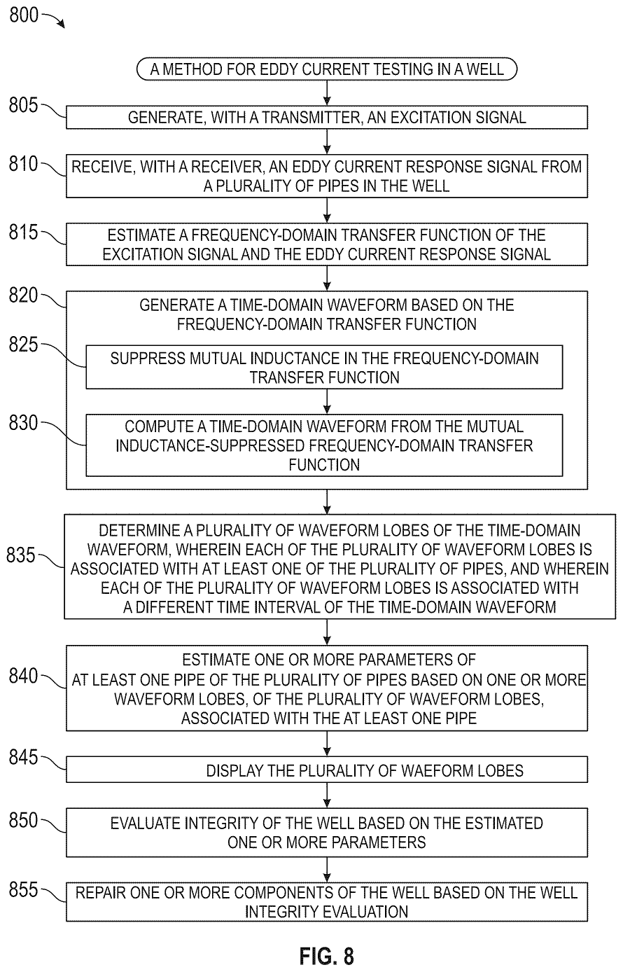

FIG. 8 depicts example operations 800 for eddy current testing and well integrity evaluation with eddy current diffusion time response. In some aspects, the well includes a nested pipe configuration.

The operations 800 may begin, at operation 805, with a transmitter generating an excitation signal. In some aspects, the excitation signal is a multi-sine, chirp, step current, or pulse signal. In some aspects, the transmitter is a driver coil. In some aspects, the transmitter excites multiple frequencies over large bandwidth. In some aspects, the transmitter excited broadband frequencies.

The operations 800 may continue, at operation 810, with a receiver receiving an eddy current response signal from a plurality of pipes in the well. In some aspects, the receiver is a pick-up coil. In some aspects, the receiver measures response voltages. In some aspects, multiple receivers are used with multiple transmitter-receiver spacings.

In some aspects, the receiver measures frequency-domain response voltages and the system generates a time-domain representation of the frequency-domain data. In some aspects, the time-domain representation of the data is time indexed.

The operations 800 may include, at operation 815, estimating a frequency-domain transfer function Z(s) (e.g., according to the Eq. 2) of the coupling between the transmitter and receiver.

The operations 800 may include, at operation 820, generating a time-domain waveform based on the frequency-domain transfer function (e.g., an eddy current diffusion time response).

Determining the time-domain waveform at operation 820 may include, at operation 825, suppressing mutual inductance in the transfer function. In some aspects, the mutual inductance is suppressed by dividing the transfer function by a complex frequency variable (e.g., s) and taking a derivation with respect of s of the remainder (e.g., according to the Eq. 6).

Determining the eddy current diffusion time response at operation 820 may include, at operation 830, computing a time-domain waveform w(t) by applying by multiplying time by the step response of the transfer function (e.g., according to the Eqs. 8-9). The time-domain waveform w(t) removes the disturbance of inductive direct coupling of the transfer function. Further, the shape of the time-domain waveform w(t) is independent of electrical conductivity of the material under inspection.

The operations 800 may include, at operation 835, determining a plurality of waveform lobes (e.g., first lobe 620, second lobe 630, third lobe 640) of the time-domain waveform. Each waveform lobe is associated with at least one of the plurality of pipes and with a different time interval of the time-domain waveform.

In some aspects, the time-domain waveform is modeled as a mixture of the plurality of waveform lobes. In some aspects, the time-domain waveform is modeled as a sum, a weighted sum, or a convolution of the plurality of waveform lobes. In some aspects, each respective waveform lobe of the plurality of waveform lobes is a function of: a thickness of the respective pipe associated with the respective waveform lobe; a thickness of any pipe, of the plurality of pipes, nested within the respective pipe; electrical conductivity of each pipe of the plurality of pipes; and magnetic permeability of each pipe of the plurality of pipes.

The operations 800 may include, at operation 840, estimating one or more parameters of interest of at least one pipe based on the generated time-domain waveform. In some aspects, the parameters of interest may include the thickness, electrical conductivity, magnetic permeability, and/or geometry of the material under inspection.

The operations 800 may include, at operation 845, determining one of sum of lobes waveforms. In some aspects, each sum of lobes waveform corresponds to a sum of multiple waveform lobes of the plurality of waveform lobes.

The operations 800 may include, at operation 845, displaying the plurality of waveform lobes.

The operations 800 may include, at operation 850, evaluating integrity of the well based on the one or more parameters. In some aspects, evaluating the integrity of the well includes identifying (e.g., and/or quantifying) cracks, corrosion, fatigue, pitting, erosion, thinning, wear, defects in one or more components of the well (e.g., pipe, coatings, welds, tubing, casing, drilling equipment, etc.). In some aspects, evaluating the well integrity includes identifying corrosion of a pipe of a nested pipe configuration based on identifying, using a displayed image, a shift in the response waveform to earlier time indexes, a shift of the response waveform increasing in amplitude, a time region of where the shift occurs, and/or a transmitter-receiver spacing at which the shift occurs.

The operations 800 may include, at operation 855, repairing one or more components of the well based on the well integrity evaluation.

### Example System for Well Integrity Evaluation with Eddy Currents

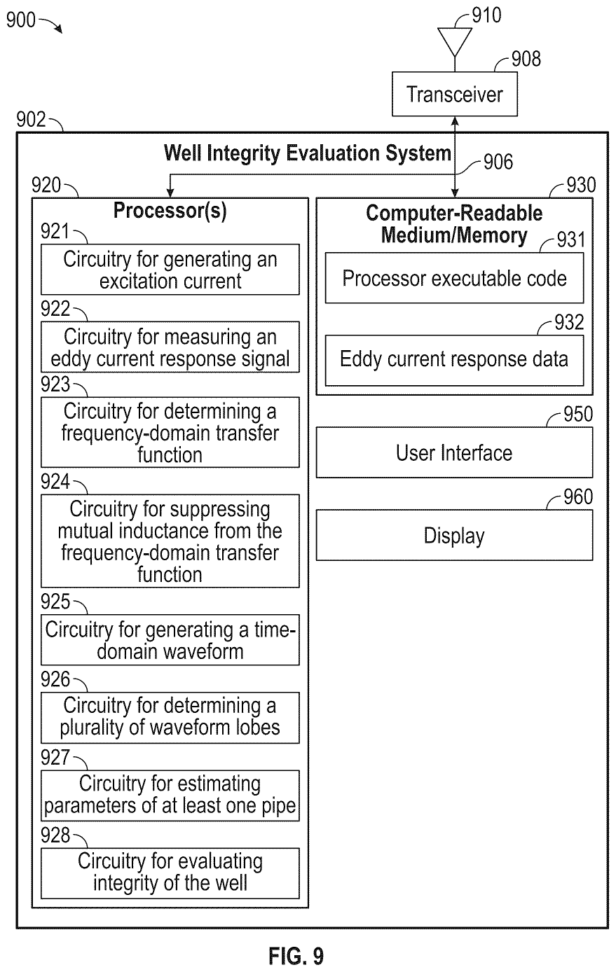

FIG. 9 depicts aspects of an example well integrity evaluation system 900. Well integrity evaluation system 900 may be implemented as a single device or, in some aspects, components of well integrity evaluation system 900 may be implemented across multiple physical devices. In some aspects, the components of well integrity evaluation system 900 may be located at the well, remotely from the well, and/or distributed across locations at the well and remote locations.

The well integrity evaluation system 900 includes a processing system 902, which may be coupled to a transceiver 908 (e.g., a transmitter and/or a receiver). The transceiver 908 may be configured to transmit and receive signals for the well integrity evaluation system 900 wirelessly via an antenna 910 or over a wired connection. The transceiver may be used for inter-communication between components within the well integrity evaluation system 900 and/or for communication with other devices or systems over a network.

The processing system 902 includes one or more processors 920. The one or more processors 920 may be coupled to a computer-readable medium/memory 930 via a bus 906 or may communicate with the computer-readable medium/memory 930 via a wired or wireless connection over a network. In certain aspects, the computer-readable medium/memory 930 is configured to store instructions (e.g., computer-executable code 731) that when executed by the one or more processors 920, cause the one or more processors 920 to perform the operations 800 described with respect to FIG. 8, or any aspect related to it. Note that reference to a processor performing a function of system 900 may include one or more processors performing that function of system 900.

The one or more processors 920 include circuitry configured to implement (e.g., execute) the aspects described herein for well integrity evaluation, including circuitry for generating an excitation current 921, circuitry for measuring an eddy current response signal 922, circuitry for determining a frequency-domain transfer function 923, circuitry for suppressing mutual inductance from the frequency-domain transfer function 924, circuitry for generating a time-domain waveform 925, circuitry for determining a plurality of waveform lobes of the time-domain waveform, circuitry for estimating parameters of at least one pipe 927, and circuitry for evaluating integrity of the well 928. Processing with circuitry 921-928 may cause the well integrity evaluation system 900 to perform the operations 800 described with respect to FIG. 8 or any aspect related to it.

Computer-readable medium/memory 930 may store information that can be used by the processors 920. For example, computer-readable medium/memory 930 may store an eddy current response data 932 measured by one or more receivers.

The well integrity evaluation system 900 may include a user interface 950 to accept inputs from a user. In some aspects, the user interface 950 is a graphical user interface (GUI). In some aspects, the GUI accepts touch screen inputs from the user. In some aspects, the user interface 950 includes one or more input/output (IOs) interfaces that allows one or more I/O devices (e.g., keyboards, displays, mouse devices, pen inputs, microphones, etc.) to connect to the well integrity evaluation system 900. The inputs from the user may include decisions on the repairing components of the well.

The well integrity evaluation system 900 may include a display 960 configured to display visualizations of the eddy current response data discussed herein, such as the plurality of waveform lobes.

### Example Clauses

Implementation examples are described in the following numbered clauses:
Clause 1: A system for eddy current testing in a well, the system comprising: one or more transmitter coils in the well configured to generate an excitation current; one or more receiver coils in the well configured to measure an eddy current response signal from a plurality of pipes in the well, wherein the eddy current response signal comprises a voltage response; and one or more processors configured to: determine a frequency-domain transfer function based on a ratio of the voltage response to the excitation current; generate a time-domain waveform based on the frequency-domain transfer function; determine a plurality of waveform lobes of the time-domain waveform, wherein each of the plurality of waveform lobes is associated with at least one of the plurality of pipes, and wherein each of the plurality of waveform lobes is associated with a different time interval of the time-domain waveform; and estimate one or more parameters of at least one pipe of the plurality of pipes based on one or more waveform lobes, of the plurality of waveform lobes, associated with the at least one pipe.
Clause 2: The system of any combination of Clause 1, wherein the one or more processors are further configured to estimate at least one of: a thickness, an electrical conductivity or an electrical permeability of the at least one pipe based on the one or more waveform lobes.
Clause 3: The system of any combination of Clauses 1-2, wherein the time-domain waveform is modeled as a mixture of the plurality of waveform lobes.
Clause 4: The system of Clause 3, wherein the time-domain waveform is modeled as a sum, a weighted sum, or a convolution of the plurality of waveform lobes.
Clause 5: The system of any combination of Clauses 1-4, wherein each respective waveform lobe of the plurality of waveform lobes is a function of: a thickness of a respective pipe of the plurality of pipes; a thickness of any pipe, of the plurality of pipes, nested within the respective pipe; electrical conductivity of each pipe of the plurality of pipes; and magnetic permeability of each pipe of the plurality of pipes.
Clause 6: The system of Clause 5, wherein the one or more processors are further configured to determine a plurality of sum of lobes waveforms, wherein each sum of lobes waveform corresponds a plurality of summed waveform lobes of the plurality of waveform lobes.
Clause 7: The system of any combination of Clauses 1-6, further comprising a display configured to display the plurality of waveform lobes.
Clause 8: The system of any combination of Clauses 1-7, wherein the one or more processors are configured to evaluate integrity of the well based on the estimated one or more parameters of the at least one pipe by identifying at least one of: cracks, corrosion, fatigue, pitting, erosion, thinning, wear, or defects in one or more components of the well.
Clause 9: A method for operating the system in accordance with any of Clauses 1-8.
Clause 10: A computer-readable medium comprising executable instructions that, when executed by one or more processors of an apparatus, cause the apparatus to operate the system in accordance with any of Clauses 1-9.
Clause 11: An apparatus comprising: a memory comprising executable instructions and one or more processors configured to execute the executable instructions and cause the apparatus to operate the system in accordance with any of Clauses 1-8.
Clause 12: An apparatus comprising means for operating the system in accordance with any of Clauses 1-8.

### Additional Considerations

The preceding description is provided to enable any person skilled in the art to practice the various aspects described herein. The examples discussed herein are not limiting of the scope, applicability, or aspects set forth in the claims. Various modifications to these aspects will be readily apparent to those skilled in the art, and the general principles defined herein may be applied to other aspects. For example, changes may be made in the function and arrangement of aspects discussed without departing from the scope of the disclosure. Various examples may omit, substitute, or add various procedures or aspects as appropriate. For instance, the methods described may be performed in an order different from that described, and various actions may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

The various illustrative logical blocks, modules and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, a system on a chip (SoC), or any other such configuration.

As used herein, “a processor,” “at least one processor” or “one or more processors” generally refers to a single processor configured to perform one or multiple operations or multiple processors configured to collectively perform one or more operations. In the case of multiple processors, performance of the one or more operations could be divided amongst different processors, though one processor may perform multiple operations, and multiple processors could collectively perform a single operation. Similarly, “a memory,” “at least one memory” or “one or more memories” generally refers to a single memory configured to store data and/or instructions, multiple memories configured to collectively store data and/or instructions.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more actions for achieving the methods. The method actions may be interchanged with one another without departing from the scope of the claims. In other words, unless a specific order of actions is specified, the order and/or use of specific actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system for eddy current testing in a well, the system comprising:
one or more transmitter coils in the well configured to generate an excitation current;
one or more receiver coils in the well configured to measure an eddy current response signal from a plurality of pipes in the well, wherein the eddy current response signal comprises a voltage response; and
one or more processors configured to:
determine a frequency-domain transfer function based on a ratio of the voltage response to the excitation current;
generate a time-domain waveform based on the frequency-domain transfer function;
determine a plurality of waveform lobes of the time-domain waveform, wherein each of the plurality of waveform lobes is associated with at least one of the plurality of pipes, and wherein each of the plurality of waveform lobes is associated with a different time interval of the time-domain waveform; and
estimate one or more parameters of at least one pipe of the plurality of pipes based on one or more waveform lobes, of the plurality of waveform lobes, associated with the at least one pipe.

2. The system of claim 1, wherein the one or more processors are further configured to estimate at least one of: a thickness, an electrical conductivity, or an electrical permeability of the at least one pipe based on the one or more waveform lobes.

3. The system of claim 1, wherein the time-domain waveform is modeled as a mixture of the plurality of waveform lobes.

4. The system of claim 3, wherein the time-domain waveform is modeled as a sum, a weighted sum, or a convolution of the plurality of waveform lobes.

5. The system of claim 1, wherein each respective waveform lobe of the plurality of waveform lobes is a function of: a thickness of a respective one of the plurality pipes; a thickness of any pipe, of the plurality of pipes, nested within the respective pipe; electrical conductivity of each pipe of the plurality of pipes; and magnetic permeability of each pipe of the plurality of pipes.

6. The system of claim 5, wherein the one or more processors are further configured to determine a plurality of sum of lobes waveforms, wherein each sum of lobes waveform corresponds a plurality of summed waveform lobes of the plurality of waveform lobes.

7. The system of claim 1, further comprising a display configured to display the plurality of waveform lobes.

8. The system of claim 1, wherein the one or more processors are configured to evaluate integrity of the well based on the estimated one or more parameters of the at least one pipe by identifying at least one of: cracks, corrosion, fatigue, pitting, erosion, thinning, wear, or defects in one or more components of the well.

9. A method for eddy current testing in a well, the method comprising:
generating, via one or more transmitter coils in the well, an excitation current;
measuring, via one or more receiver coils in the well, an eddy current response signal from a plurality of pipes in the well, wherein the eddy current response signal comprises a voltage response;
determining, via one or more processors, a frequency-domain transfer function based on a ratio of the voltage response to the excitation current;
generating, via the one or more processors, a time-domain waveform based on the frequency-domain transfer function;
determining, via the one or more processors, a plurality of waveform lobes of the time-domain waveform, wherein each of the plurality of waveform lobes is associated with at least one of the plurality of pipes, and wherein each of the plurality of waveform lobes is associated with a different time interval of the time-domain waveform; and
estimating, via the one or more processors, one or more parameters of at least one pipe of the plurality of pipes based on one or more waveform lobes, of the plurality of waveform lobes, associated with the at least one pipe.

10. The method of claim 9, further comprising estimating, via the one or more processors, at least one of: a thickness, an electrical conductivity, or an electrical permeability of the at least one pipe based on the one or more waveform lobes.

11. The method of claim 9, further comprising modeling the time-domain waveform as a mixture of the plurality of waveform lobes.

12. The method of claim 11, wherein the time-domain waveform is modeled as a sum, a weighted sum, or a convolution of the plurality of waveform lobes.

13. The method of claim 9, wherein each respective waveform lobe of the plurality of waveform lobes is a function of: a thickness of a respective one of the plurality of pipes; a thickness of any pipe, of the plurality of pipes, nested within the respective pipe; electrical conductivity of each pipe of the plurality of pipes; and magnetic permeability of each pipe of the plurality of pipes.

14. The method of claim 13, further comprising determining a plurality of sum of lobes waveforms, wherein each sum of lobes waveform corresponds a plurality of summed waveform lobes of the plurality of waveform lobes.

15. The method of claim 9, further comprising displaying the plurality of waveform lobes.

16. The method of claim 9, further comprising evaluating integrity of the well based on the estimated one or more parameters of the at least one pipe by identifying at least one of: cracks, corrosion, fatigue, pitting, erosion, thinning, wear, or defects in one or more components of the well.

17. The method of claim 16, further comprising initiating a repair or replacement of at least one of the one or more components of the well based on the evaluation of the integrity of the well.

18. A non-transitory computer readable medium comprising computer executable code for eddy current testing in a well, the computer executable code comprising:
code for determining a frequency-domain transfer function based on a ratio of an eddy current voltage response signal to an excitation current;
code for generating a time-domain waveform based on the frequency-domain transfer function;
code for determining a plurality of waveform lobes of the time-domain waveform, wherein each of the plurality of waveform lobes is associated with at least one of a plurality of pipes, and wherein each of the plurality of waveform lobes is associated with a different time interval of the time-domain waveform; and
code for estimating one or more parameters of at least one pipe of the plurality of pipes based on one or more waveform lobes, of the plurality of waveform lobes, associated with the at least one pipe.

19. The non-transitory computer readable medium of claim 18, further comprising code for estimating, via the one or more processors, at least one of: a thickness, an electrical conductivity, or an electrical permeability of the at least one pipe based on the one or more waveform lobes.

20. The non-transitory computer readable medium of claim 18, further comprising code for modeling the time-domain waveform as a mixture of the plurality of waveform lobes.
